(19) United States
(12) Patent Application Publication
(10) Pub. No.: US 2010/0022777 A1
(43) Pub. Date: Jan. 28, 2010

(54) Process for the Preparation of E-3-[2-(7-Chloro-2-Quinolinyl)Ethenyl]Benzaldehyde

(75) Inventors: Sugata Chatterjee, District Vadodara (IN); Ajay Singh Rawat, District Vadodara (IN); Neeraj Kumar, District Vadodara (IN); Mukesh Chavda, District Vadodara (IN); Rushikesh Kadu, District Vadodara (IN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sterling Biotech Limited, Mumbai (IN)

(21) Appl. No.: 12/260,427

(22) Filed: Oct. 29, 2008

(30) Foreign Application Priority Data
Jul. 23, 2008 (IN) 1564/MUM/2008

(51) Int. Cl. C07D 215/18

(52) U.S. Cl. 546/180

(57) ABSTRACT

An improved, scalable, and environmentally friendly manufacturing procedure for the preparation of E-3-[2-(7-chloro-2-quinolinyl)ethenyl]benzaldehyde, which is a key early intermediate used in the preparation of montelukast sodium, and the compound prepared by such a process.

### CROSS-REFERENCES TO RELATED APPLICATIONS

This application claims benefits of Indian Patent Application No. 1564/Mum/2008 filed, Jul. 23, 2008, the contents of which is incorporated herein by reference in its entirety.

### FIELD OF THE INVENTION

The invention relates to an improved, scalable, and environmentally friendly manufacturing procedure for the preparation of E-3-[2-(7-chloro-2-quinolinyl)ethenyl]benzaldehyde, which is a key early intermediate used in the preparation of Montelukast sodium.

Montelukast sodium (SINGULAIR®) is described chemically as [R-(E)]-[[[1-[3-[2-(7-chloro-2-quinolinyl)ethenyl]phenyl]-3-[2-(1-hydroxy-1-methylethyl)phenyl]propyl]thio]methyl]cyclopropane acetic acid sodium. It is a selective and orally active leukotriene receptor anatagonist that inhibits the cysteinyl leukotriene CysL T₁ receptor and is useful in the treatment of asthma as well as other conditions mediated by leukotrienes like prevention of exercise induced bronchospasm. Montelukast is also used for the symptomatic treatment of seasonal or perennial allergic rhinitis and has also been evaluated for the management of urticaria.

### BACKGROUND OF THE INVENTION

Montelukast sodium a leukotriene antagonist useful in the treatment of asthma and related problems is described as [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropane acetic acid sodium and has the following structural formula I:

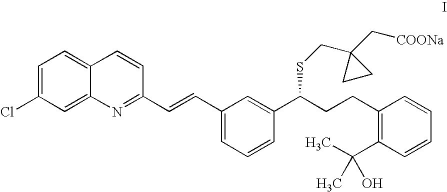

### Related Art

The complete synthesis of Montelukast sodium is described in European publication 0480717. This patent does not mention the process for the preparation of 3-[2-(7-chloro-2-quinolinyl)ethenyl]benzaldehyde, which is the first key intermediate in the synthesis of Montelukast sodium and represented by the following structural formula II,

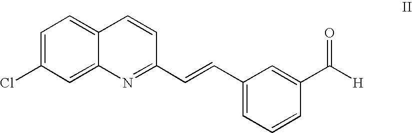

but provides the cross reference U.S. Pat. No. 4,851,409 by Young et al. In this patent, formula II is reported to be synthesized by reacting 1.0 molar equivalent of isophthaldehyde with 1.08 molar equivalents of 7-chloroquinaldine in acetic anydride at 125° C. for 48.0 hrs and the crude product isolated via filtration after precipitation with ether. The discussed method has the following drawbacks:

The reaction uses acetic anhydride, which is a regulated commercial item as it is rampantly used for making banned narcotic drugs, hence making the process commercially unviable and unsafe.

Since formula II is an early intermediate, the amount of acetic anhydride to be used is large. Use of acetic anhydride on large scale is very much inconvenient due to all the hazards associated with it (page 19 of Sax's 11th edition, Dangerous Properties of Industrial Materials, by Richard J. Lewis, Sr., Volume 2).

The reaction time is long.

Isolation step involves precipitation by the addition to the reaction mixture of ether, the use of which is not safe and economical for large scale manufacturing, due its high volatility and flammability.

Further the disclosed process does not mention the quality and yield of the obtained intermediate.

Another patent application GB 2322624 A describes the process for the preparation of this key intermediate. Herein the reaction of 7-chloroquinalidine and isophthaldehyde is performed in the presence of acetic anhydride in a binary mixture of toluene and n-heptane in a preferred ratio of 25:75 at 99-101° C. for 13.0 hours. However, this described process suffers from the following major drawbacks:

Firstly, this discussed process also uses acetic anhydride and thus carries the associated drawbacks mentioned above.

The reaction is carried out in a binary mixture of toluene and n-heptane making the process uneconomical when scaled up.

Also as per the discussed process, excess of the binary solvent mixture (toluene/n-heptane) is added after completion of reaction as a filtration aid thus increasing the effluent load.

The discussed process does not disclose the recyclability of the used binary solvent system.

The discussed process emphasizes only the yield and does not mention the quality.

The patent also exhibits examples in which reactions are performed in xylene and dry n-butyl acetate, which may not be economically viable to be employed for manufacturing purposes.

WO 2006/021974 describes the reaction of 7-Chloroquinalidine with isophthaldehdye in the presence of acetic anhydride in toluene at 100-105° C. for 12-15 hours. This process has the following shortcomings:

Firstly, the described process also uses acetic anhydride, which accompanies the associated drawbacks mentioned earlier.

After completion of the reaction, excess n-hexane is added to the reaction mixture to facilitate precipitation and filtration creating a binary solvent mixture of toluene and hexane, an undesirable effluent load.

Also, the described process does not teach the fate of the used toluene and hexane mixture, thus making the process uneconomical and eco-unfriendly for large scale manufacturing.

### BRIEF SUMMARY OF THE INVENTION

The invention overcomes the drawbacks as encountered in the prior art and provide an improved, scalable, efficient, and eco-friendly manufacturing procedure for the preparation of 3-[2-(7-chloro-2-quinolnyl)ethenyl]benzaldehyde, a key initial intermediate used in the preparation of Montelukast sodium.

The invention provides an improved, scalable, and efficient manufacturing procedure for the preparation of 3-[2-(7-chloro-2-quinolinyl)ethenyl]benzaldehyde, a key intermediate used in the preparation of Montelukast Sodium, wherein the reaction is performed in a suitable single organic solvent.

The invention also provides an improved, scalable and efficient manufacturing procedure for the preparation of 3-[2-(7-chloro-2-quinolinyl)ethenyl]benzaldehyde a key intermediate used in the preparation of Montelukast Sodium, wherein the reaction is performed without acetic anhydride.

The invention also provides an improved, scalable, and efficient manufacturing procedure for the preparation of 3-[2-(7-chloro-2-quinolinyl)ethenyl]benzaldehyde, a key intermediate used in the preparation of Montelukast Sodium, wherein the reaction is performed in the presence of a suitable base, acid, or a combination thereof.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### BRIEF DESCRIPTION OF THE FIGURES

The accompanying figures, which are included to provide a further understanding of the invention, are incorporated in and constitute a part of this specification, illustrate embodiments of the invention and together with the detailed description serve to explain the principles of the invention. No attempt is made to show details of the invention in more detail than may be necessary for a fundamental understanding of the invention and various ways in which it may be practiced.

FIG. 1: ¹H-NMR of 3-[2-(7-chloro-2-quinolinyl)-E-ethenyl]benzaldehyde recorded at 500 MHz in CDCl₃.

FIG. 2: FT-IR spectrum of 3 -[2-(7-chloro-2-quinolinyl)-E-ethenyl]benzaldehyde.

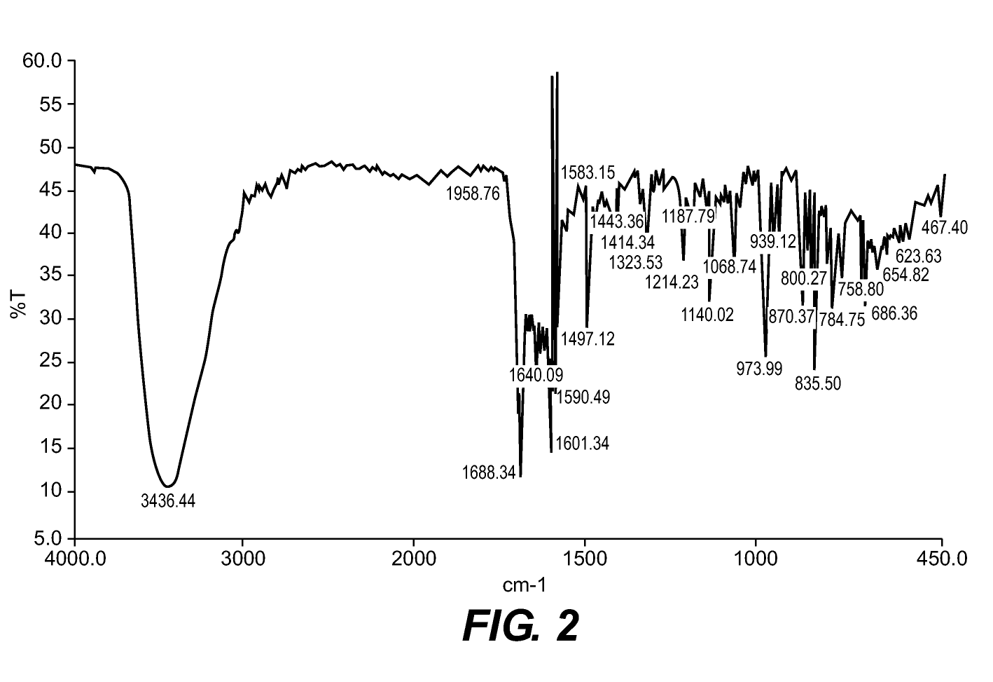

FIG. 3: ¹H-NMR of the separated bis adduct impurity V recorded at 500 MHz in DMSO-d₆ and trace DCl.

FIG. 4: FT-IR spectrum of the separated bis adduct impurity V.

The objectives as mentioned above will be apparent in the following detailed description.

### DETAILED DESCRIPTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a reagent” is a reference to one or more reagents and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying figures and detailed in the following description. It should be noted that the features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

The process of the invention is illustrated in scheme I and is described below.

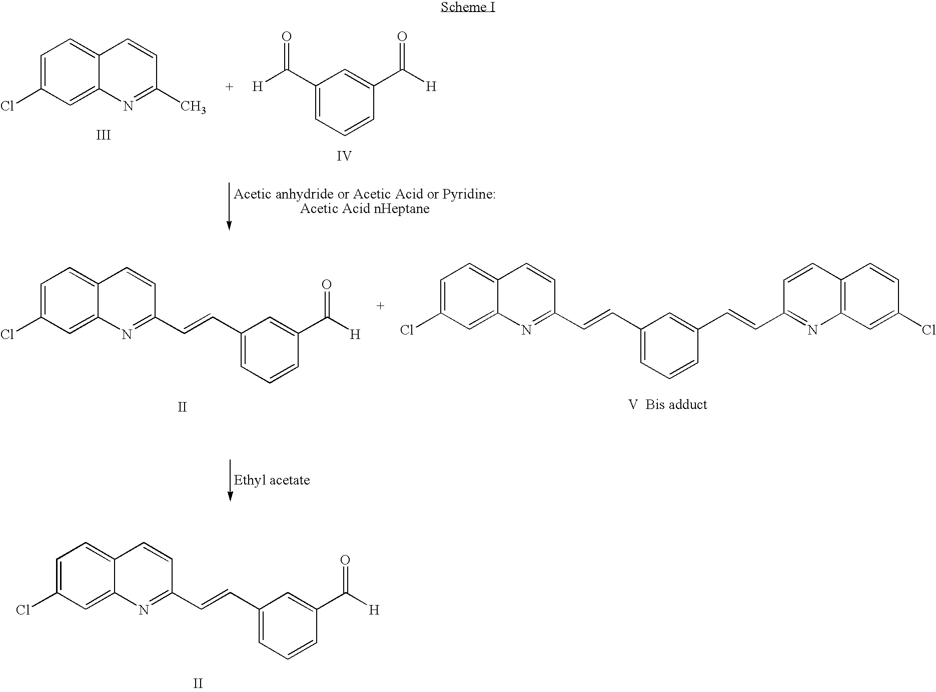

Accordingly, the invention provides an improved, scalable, and efficient manufacturing procedure for the preparation of 3-[2-(7-chloro-2-quinolinyl)ethenyl]benzaldehyde, a key initial intermediate used in the preparation of Montelukast sodium, the process comprising the steps of:
(1) Reacting 7-chloroquinaldine (III) and isophthaldehyde (IV) in the presence of suitable base, acid, or a combination thereof optionally in a suitable organic solvent or without solvent;
(2) Isolating the crude intermediate by direct filtration from the reaction mass;
(3) Purifying the crude intermediate;
(4) Recovering the organic solvent from mother liquor by atmospheric or vacuum distillation, if so desired, and using the recovered solvent for the said reaction.

According to the invention, the suitable single organic solvent for the reaction may be a linear or branched C5 to C10 hydrocarbon, e.g., an aliphatic compound, such as n-pentane, n-hexane, hexanes, n-heptane, heptanes, n-octane etc., or an aromatic hydrocarbons such as toluene, xylene, etc. The most preferred solvent is n-heptane.

The invention further provides a process wherein the reaction is carried out at any temperature between ambient and reflux temperature of the chosen organic solvent.

The invention further provides a process wherein 0.9 to 3.0 molar equivalent of isophthaldehyde with respect to 7-chloroquinaldine is used in the reaction. The preferred amount of isophthaldehyde to be used is 0.98 to 1.50 molar equivalent, most preferably 1.30 to 1.50 molar equivalent.

The invention further provides a process wherein the suitable base is chosen from pyridine, piperidine, triethylamine, diisopropylethylamine, etc., most preferably pyridine.

The invention also provides a process wherein the suitable acid is chosen from acetic acid, oxalic acid, tartaric acid, paratoluenesulphonic acid, and methanesulphonic acid, etc., most preferably acetic acid.

The invention further provides a process wherein the suitable acid base combination is chosen from triethylanimonium acetate, triethylammonium chloride, diisopropylethylammonium acetate, diisopropylethylammonium chloride, piperidinium acetate, piperidinium chloride, pyridinium chloride, pyridinium acetate, etc., most preferably pyridinium acetate.

Thus, the invention provides an improved manufacturing procedure for the preparation of 3-[2-(7-chloro-2-quinolinyl)ethenyl]benzaldehyde that overcomes the drawbacks of the earlier disclosed methods. It requires reduced reaction time and leads to isolation of the said intermediate in reasonable yield and quality.

The process of the invention also reduces the effluent load by using a single suitable organic solvent in place of a binary mixture of solvents as described in the prior art. The single solvent, such as, for example, n-heptane, can be recovered and reused easily for the process, hence making the whole process economical and an environmentally benign one.

The invention also provides the compound 3-[2-(7-chloro-2-quinolnyl)ethenyl]benzaldehyde prepared by the process described above.

Without further elaboration, it is believed that one skilled in the art using the preceding description can utilize the invention to the fullest extent. The following examples are illustrative only, and not limiting of the disclosure in any way whatsoever.

### EXAMPLES

The following examples (wherein no acetic anhydride has been used) illustrate, but in no way limit the scope the novel process of this invention. Any deviation from this, apparent and obvious to a person skilled in the art of organic synthesis, forms part of this invention though not explicitly substantiated.

### Example 1

A solution of 7-chloroquinaldine (5 g, 0.028 mol, 1.0 eq.), isophthaldehyde (5.63 g, 0.042 mol, 1.5 eq), pyridine (2.26 ml, 0.028 mol, 1.0 eq), and acetic acid (1.6 ml, 0.028 mol, 1.0 eq) in n-heptane (30 ml, 6 vol) was heated at reflux for 8-9 hrs. The reaction mixture was allowed to cool to room temperature. The precipitated crude solid was filtered. The wet crude product was charged to 150.0 ml ethyl acetate and heated to reflux with stirring for 2 hrs. The solution was filtered hot and was then concentrated under vacuum to ˜10.0 ml. The resultant slurry was cooled below 20° C. and stirred at the same temperature for 2 hrs. The solid was then filtered, washed with chilled ethyl acetate, and dried at 55-60° C. under vacuum for 5.0 hours. Yield 4.40 gm (55.04%), Purity (HPLC) 99.42%.

### Example 2

A solution of 7-chloroquinaldine (5.0 g, 0.028 mol, 1.0 eq), isophthaldehyde (5.63 g, 0.042 mol, 1.5 eq), pyridine (2.26 ml, 0.028 mol, 1.0 eq), and acetic acid (1.6 ml, 0.028 mol, 1.0 eq) in toluene (30 ml, 6 vol) was heated at reflux for 8-9 hrs. The reaction mixture was allowed to cool to room temperature. The precipitated crude solid was filtered. The wet crude product was charged to 150 ml ethyl acetate and heated to reflux with stirring for 2 hrs. The solution was filtered hot and then concentrated under vacuum to ˜10.0 ml. The resultant slurry was cooled below 20° C. and stirred at same temperature for 2 hrs. The solid was then filtered, washed with chilled ethyl acetate, and dried at 55-60° C. under vacuum for 5.0 hours. Yield 3.96 gm (48.0%), Purity (HPLC) 99.63%.

### Example 3

A solution of 7-chloroquinaldine (25 g, 0.141 mol, 1.0 eq), isophthaldehyde (28.38 g, 0.212 mol, 1.5 eq), and acetic acid (8.4 ml, 0.141 mol, 1.0 eq) in n-heptane (150 ml, 6 vol) was heated at reflux for 6-7 hrs. The reaction mixture was allowed to cool to room temperature. The precipitated crude solid was filtered. The wet crude product was charged to 750.0 ml ethyl acetate and heated to reflux with stirring for 2 hrs. The solution was filtered in hot and was then concentrated under vacuum to ˜50.0 ml. The resultant slurry was cooled below 20° C. and stirred at same temperature for 2 hrs. The solid was then filtered, washed with chilled ethyl acetate, and dried at 55-60° C. under vacuum for 5.0 hours. Yield 26.3 gm (63.75%), Purity (HPLC) 99.84%.

### Example 4

A solution of 7-chloroquinaldine (25 g, 0.141 mol, 1.0 eq), isophthaldehyde (28.38 g, 0.212 mol, 1.5 eq), and acetic acid (8.4 ml, 0.141 mol, 1.0 eq) in toluene (150 ml, 6 vol) was heated at reflux for 6-7 hrs. The reaction mixture was allowed to cool to room temperature. The precipitated crude solid was filtered. The wet crude product was charged to 750.0 ml. ethyl acetate and heated to reflux with stirring for 2 hrs. The solution was filtered in hot and was then concentrated under vacuum to ˜50.0 ml. The resultant slurry was cooled below 20° C. and stirred at same temperature for 2 hrs. The solid was then filtered, washed with chilled ethyl acetate, and dried at 55-60° C. under vacuum for 5.0 hours. Yield 18.56 gm (45.0%), Purity (HPLC) 99.89%.

### Example 5

A solution of 7-chloroquinaldine (100 g, 0.565 mol, 1.0 eq), isophthaldehyde (113.54 g, 0.847 mol, 1.5 eq), and acetic acid (200 ml, 2 vol) were heated at 100° C. for 1-2 hr. The reaction mixture was allowed to cool to room temperature. The precipitated crude solid was filtered and washed with 200 ml methanol. The wet crude product was charged to 3000 ml ethyl acetate and heated to reflux with stirring for 2 hrs. The solution was filtered in hot and was then concentrated under vacuum to ˜200.0 ml. The resultant slurry was cooled below 20° C. and stirred at same temperature for 2 hrs. The solid was then filtered, washed with chilled ethyl acetate, and dried at 55-60° C. under vacuum for 5.0 hours. Yield 108.2 gm (65.45%), Purity (HPLC) 99.94%.

### Example 6

A solution of 7-chloroquinaldine (5.0 g, 0.028 mol, 1.0 eq), isophthaldehyde (5.66 g, 0.042 mol, 1.5 eq), and acetic acid (0.162 ml, 0.0028 mol, 0.1 eq) in n-heptane (30 ml, 6 vol) was heated at reflux for 7-8 hrs. The reaction mixture was then allowed to cool to room temperature. The crude solid was filtered. The crude product was charged to 150 ml of ethyl acetate and heated to reflux with stirring for 2.0 hrs. The solution was filtered hot to remove the insoluble bis adduct. Filtrate was concentrated partially under vacuum, cooled, and stirred at 15 to 20° C. for 2.0 hrs. The resulting solid was then filtered, washed with chilled ethyl acetate and dried at 55-60° C. under vacuum. Yield 3.95 gm (47.80%), Purity (HPLC) 98.87%. Melting Point 149-151° C..

### Example 7

A solution of 7-chloroquinaldine (5.0 g, 0.028 mol, 1.0 eq), isophthaldehyde (5.66 g, 0.042 mol, 1.5 eq), and acetic acid (0.32 ml, 0.0056 mol, 0.2 eq) in n-heptane (30 ml, 6 vol) was heated at reflux for approximately 6.0 hrs. The reaction mixture was then allowed to cool to room temperature. The crude solid was filtered. The crude product was then charged to 150 mi of ethyl acetate and heated to reflux with stirring for 2.0 hrs. The solution was then filtered hot to remove the insoluble bis adduct. The filtrate was then concentrated partially under vacuum, cooled and stirred at 15 to 20° C. for 2.0 hrs. The resulting solid was then filtered, washed with chilled ethyl acetate and dried at 55-60° C. under vacuum. Yield 4.30 gm (52.50%); Purity (HPLC) 99.72%.

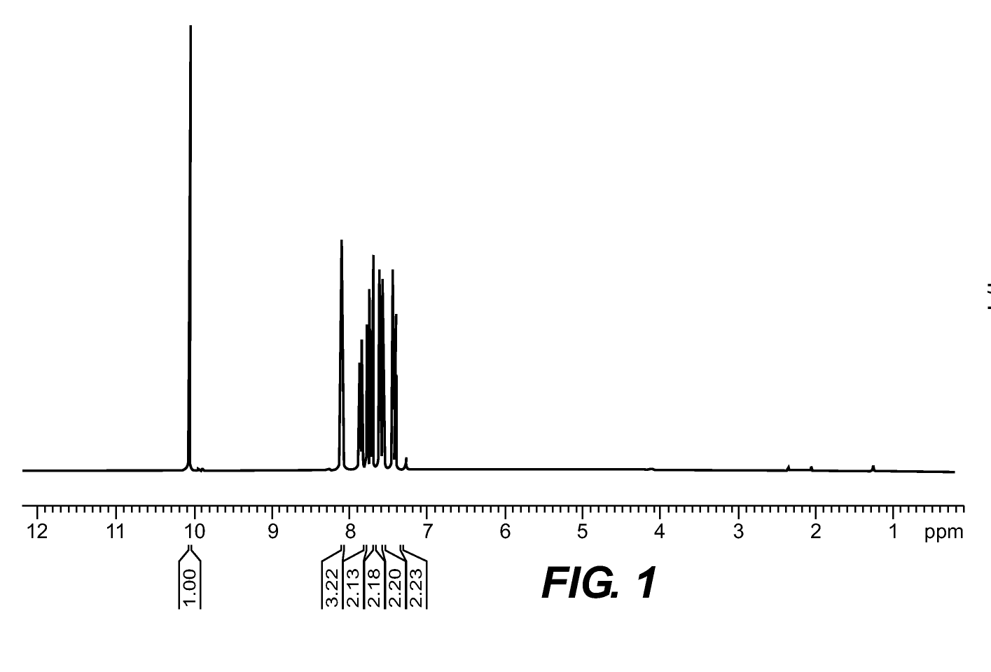

¹H-NMR (500 MHz, CDCl₃) δ 10.05 (s, 1H); 8.07-8.13 (m, 3H), 7.81-7.86 (m, 2H); 7.69-7.76 (m, 2H); 7.52-7.61 (m, 2H), 7.40-7.44 (m, 3H). [FIG. 1]

¹³C-NMR (125.76 MHz, CDCl₃) δ 192.00 (CH), 156.16 (C), 148.59 (C), 137.38 (C), 136.94 (C), 136.36 (CH), 135.74 (C), 133.47 (CH), 132.95 (CH), 130.07 (CH), 129.77 (C), 129.55 (CH), 128.69 (CH), 128.25(CH), 128.16 (CH), 127.41 (CH), 125.81 (C), 119.88 (CH).

ESI-MS: M+H⁺ ions observed at 294 amu and 295 amu.

IR νₘₐₓ(KBr) cm⁻¹: 1958, 1688, 1640, 1590, 1583, 1497, 1443, 1414, 1323, 1241, 1187, 1140, 1068, 974, 939, 870, 835, 800, 758, 686, 654. [FIG, 2]

Analytical data of the isolated bis adduct V

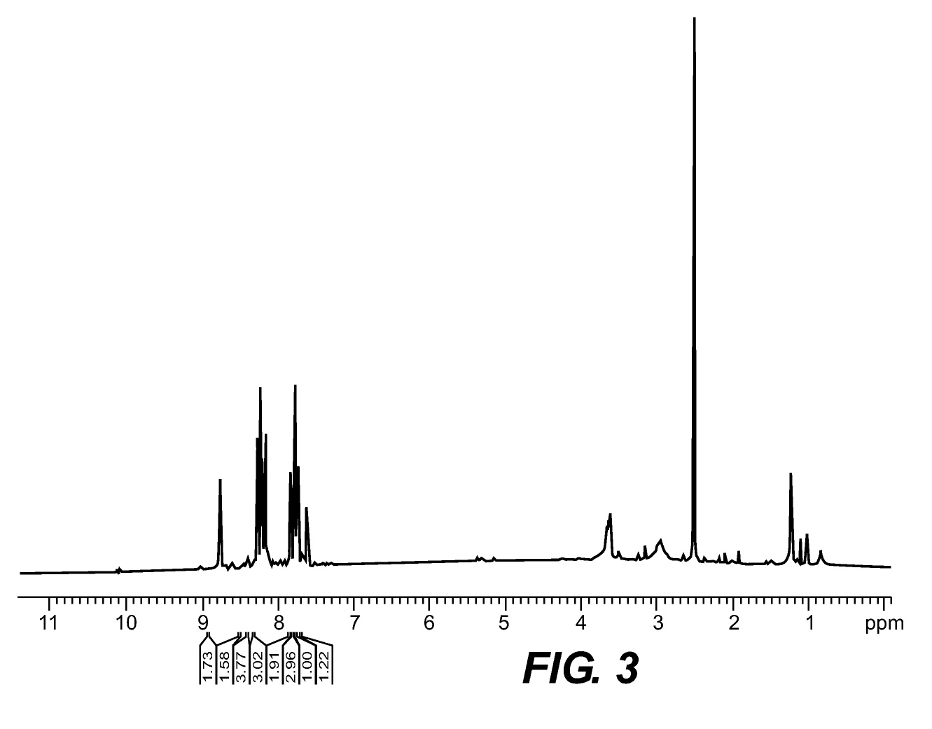

¹H-NMR (500 MHz, DMSO-d₆ with trace DCl) δ: 8.77 (d, 2H), 8.32-8.15 (m, 8H), 7.76-7.88 (m, 5H), 7.74 (s, 1H) 7.62-7.66 (m, 1H). [FIG. 3]

ESI-MS: M+H⁺ ions observed at 453 amu and 454 amu.

MS/ MS (Ar gas, collision energy 15 eV): Daughter ion peaks observed at m/z 164, 177, 277, 290, 438 amu.

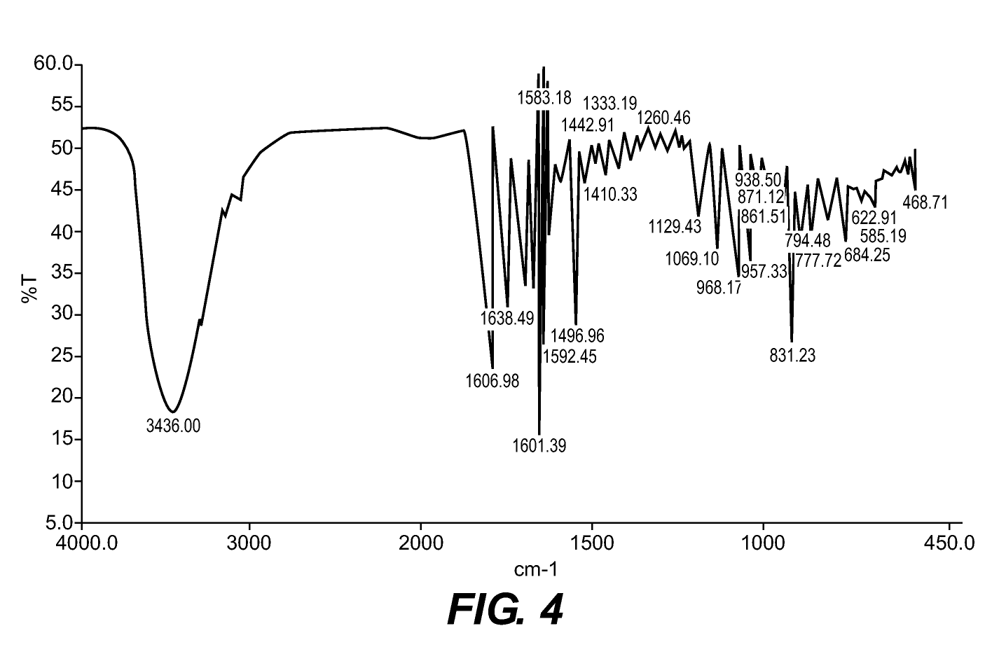

IR νₘₐₓ(KBr) cm⁻¹: 1958; 1638, 1640, 1592, 1583, 1496, 1442, 1410, 1333, 1260, 1129, 1069, 968, 957, 871, 835, 831, 794, 777, 684. [FIG. 4]

Table 2 summarizes some of the examples of the invention, while Table 1 shows the results obtained by using acetic anhydride as a reference for comparison.

The disclosures of each reference and publication cited above is expressly incorporated by reference in its entirety to the same extent as if each were incorporated by reference individually.

TABLE 1
Preparation of 3-[2-(7-chloro-2-quinolinyl)ethenyl]benzaldehyde (II) using acetic anhydride

| | | | | II | | |
|---|---|---|---|---|---|---|
| | | | | Yield | | |
| | | | Time | (Molar | HPLC | Impurity V |
| Expt. No | Chemicals | Solvent | (hrs) | %) | Purity % | (Molar %) |
| 1. | III (1.0eq.), IV (1.30eq), | n-Heptane | 6.0 | 54.41 | 98.95 | 7.03 |
| | Acetic Anhydride (1.5eq.) | | | | | |
| 2. | III (1.0eq.), IV (1.50eq), | n-Heptane | 6.0 | 54.41 | 99.58 | 7.81 |
| | Acetic Anhydride (1.5eq.) | | | | | |
| 3. | III (1.0eq.), IV (1.50eq), | Heptane-Toluene | 6-7 | 45.20 | 99.66 | 3.90 |
| | Acetic Anhydride (1.5eq.) | | | | | |
| 4. | III (1.0eq.), IV (1.50eq), | n-Heptane (recovered) | 7.0 | 65.34 | 99.60 | 8.12 |
| | Acetic Anhydride (1.5eq.) | | | | | |
| 5. | III (1.0eq.), IV (1.50eq), | Acetonitrile | 6.0 | 8.43 | 94.52 | 1.95 |
| | Acetic Anhydride (1.5eq.) | | | | | |
| 6. | III (1.0eq.), IV (1.50eq), | 1,4-Dioxane | 6.0 | 39.18 | 99.35 | 1.79 |
| | Acetic Anhydride (1.5eq.) | | | | | |
| 7. | III (1.0eq.), IV (1.50eq), | N,N-Dimethylformamide | 6-7 | 30.13 | 95.85 | 2.26 |
| | Acetic Anhydride (1.5eq.) | | | | | |
| 8. | III (1.0eq.), IV (1.50eq), | Dimethylsulfoxide | 6-7 | 36.16 | 97.68 | 1.87 |
| | Acetic Anhydride (1.5eq.) | | | | | |

TABLE 2
Preparation of 3-[2-(7-chloro-2-quinolinyl) ethenyl] benzaldehyde (II) without using acetic anhydride

| | | | | | II | | |
|---|---|---|---|---|---|---|---|
| | | | | Reaction | Yield | | Impurity V |
| Expt | | | | Time | (Molar | HPLC^{b} | (Molar |
| No. | Chemicals | Base/Acid^{a} | Solvent | (hrs) | %) | Purity % | %) |
| 1. | III (1.0eq), IV (1.30eq) | Pyridine-Acetic acid* | n-Heptane | 7.0 | 48.42 | 97.99 | 3.90 |
| | | (1.0eq) | | | | | |
| 2. | III (1.0eq), IV (1.50eq) | Pyridine-Acetic acid | n-Heptane | 7.0 | 53.04 | 99.42 | 7.81 |
| | | (1.0eq) | | | | | |
| 3. | III (1.0eq), IV (1.50eq) | Pyridine-Acetic acid | Toluene | 7.0 | 48.39 | 99.63 | 5.46 |
| | | (1.0eq) | | | | | |
| 4. | III (1.0eq), IV (1.50eq) | Acetic acid (1.0eq.) | n-Heptane | 6.0 | 63.64 | 99.84 | 8.62 |
| 5. | III (1.0eq), IV (1.50eq) | Acetic acid (1.0eq), | Toluene | 12.0 | 41.14 | 99.89 | 5.48 |
| 6. | III (1.0eq), IV (1.50eq) | Acetic acid (1.0eq), | n-Heptane- | 6.0 | 30.13 | 98.65 | 3.51 |
| | | | Toluene | | | | |
| 7. | III (1.0eq), IV (1.50eq) | Acetic acid | Acetic acid | 1.0-2.0 | 65.45 | 99.94 | 7.75 |
| 8. | III (1.0eq), IV (1.50eq) | Acetic acid (0.1eq.) | n-Heptane | 7.0-8.0 | 47.80 | 98.87 | 6.47 |
| 9. | III (1.0eq), IV (1.50eq) | Acetic acid (0.2eq.) | n-Heptane | 5.0-6.0 | 52.50 | 99.72 | 6.10 |

^{a}Commercially available glacial acetic acid ~98.0% pure by GC was used for the reaction purpose.
^{b}HPLC Conditions: Column: Waters Spherisorb 250 × 0.4 cm, 5 μm, Buffer: PBS: Methanol (30:70), pH = ~3.0, flow rate: 1.0 ml/min, Detection at 260 nm

What is claimed is:

1. A process for the preparation of 3-[2-(7-chloro-2-quinolinyl)ethenyl]benzaldehyde (II) 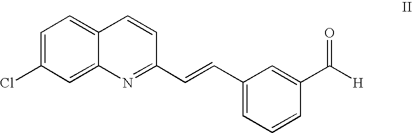
comprising:
(a) reacting 7-chloroquinaldine (III) with isopthaldehyde (IV) in the presence of a suitable base, acid, or a combination thereof in a single organic solvent 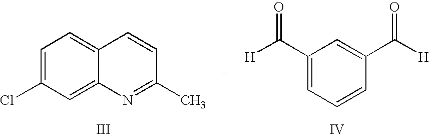
to produce a mixture of crude compounds II and V: 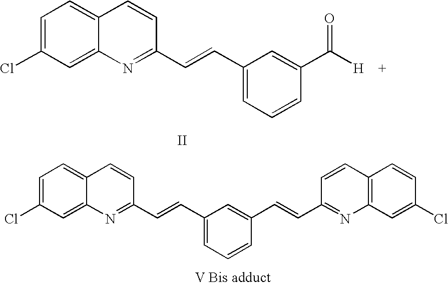
(b) isolating the crude compounds by filtration, and
(c) purifying the crude compounds to remove the bis adduct of formula V by crystallization from an organic solvent.

2. The process of claim 1, wherein 0.90 to 3.0 molar equivalent of isopthaldehyde is used.

3. The process of claim 1, wherein the reaction is performed without using acetic anhydride.

4. The process of claim 1, wherein the reaction is carried out in the presence of a suitable base.

5. The process of claim 4, wherein the suitable base is pyridine is the base.

6. The process of claim 1, wherein the reaction is carried out in the presence of an acid.

7. The process of claim 6, wherein the acid is acetic acid.

8. The process of claim 7, wherein acetic acid is the acid as well as the organic solvent for the reaction.

9. The process of claim 1, wherein the reaction is carried out in the presence of a combination of a base and acid.

10. The process of claim 9, wherein the combination of a base and acid is triethylammonium acetate, triethylammonium chloride, diisopropylammonium acetate, diisopropylammonium chloride, piperidinium acetate, piperidinium chloride, pyridinium. chloride, or pyridinium acetate.

11. The process of claim 10, wherein the combination of base and acid is pyridinium acetate.

12. The process of claim 1, wherein the organic solvent for the reaction is a C5 to C10 linear or branched aliphatic compound or an aromatic hydrocarbon.

13. The process of claim 12, wherein the organic solvent for the reaction is n-heptane.

14. The process of claim 12, wherein the organic solvent for the reaction is heptanes.

15. The process of claim 1, wherein the reaction is carried out at ambient to reflux temperature of the organic solvent for the reaction.

16. The compound 3-[2-(7-chloro-2-quinolinyl)ethenyl]benzaldehyde (II) prepared by the process of claim 1.
